(19) United States
(12) Patent Application Publication
(10) Pub. No.: US 2003/0093126 A1
(43) Pub. Date: May 15, 2003

(54) Implantable heart stimulator

(76) Inventors: Martin Obel, Danderyd (SE); Budgifvars Goran, Spanga (SE); Asa Uhrenius, Stockholm (SE)

Correspondence Address: SCHIFF HARDIN & WAITE, 6600 SEARS TOWER, 233 S WACKER DR, CHICAGO, IL, 60606-6473, US

(21) Appl. No.: 10/239,970

(22) PCT Filed: Mar. 28, 2001
(86) PCT No.: PCT/SE01/00680

(30) Foreign Application Priority Data
Mar. 29, 2000 (SE) 0001113-0

(51) Int. Cl. A61N 1/37

(52) U.S. Cl. 607/27

(57) ABSTRACT

Implantable heart stimulator comprising a pulse generating means 4 for generating stimulation pulses adapted to be applied to a heart via at least one electrode lead 6, a control means 8 with threshold search means 10 for initiating a stimulation threshold search in order to determine a stimulation threshold of heart tissue. The control means further comprises threshold search timing means 12 that generates a timing signal at same time(s) every day in order to initiate threshold searches provided that a stable condition index fulfils predetermined criteria. The stable condition index is related to the condition of the heart and is based on result from at least one heart stability test.

### BACKGROUND OF THE INVENTION

1. Field of the Invention

The present invention relates to an implantable heart stimulator of the type wherein threshold searching is undertaken at regular time intervals, and wherein, before undertaking a threshold search, a determination is made as to whether a stable condition index fulfills at least one predetermined criterion and wherein, if the stable condition index does not meet the at least one predetermined criterion, the threshold search either is not undertaken, or is postponed.

2. Description of the Prior Art

Immediately after implantation of a heart stimulator and insertion of an heart electrode lead into the heart and attaching a stimulation electrode to heart tissue, the stimulation threshold, i.e. the least energy (pulse amplitude if the pulse width is constant) required to achieve heart contraction, is relatively high. After the first months following implantation of a heart stimulator the stimulation threshold eventually stabilizes at a more or less constant value in the order of some Volts (1-5 Volts).

Natural fluctuations of the stimulation threshold occur due to e.g. the activity of the patient (awake or asleep), the intake of drugs, etc.

A threshold search algorithm is implemented in many pacemakers of today that performs threshold searches either in response of one or many losses of capture or at timely intervals, e.g. 8 hours since the last threshold search was performed.

FIG. 1 shows an IEGM illustrating the principles of a threshold search algorithm according to established standard prior art, see e.g. U.S. Pat. No. 5,476,487, and applicable in relation to the present invention. A and V designate atrial and ventricular stimulation pulses, respectively. BU is a high output backup pulse delivered if loss of capture (LOC) occurs. As can be seen (complex 3) the pre-programmed AV-interval is prolonged by Δ when a LOC occurs (complex 2). The reason for that is to await any intrinsic event if the first LOC was the result of a fusion beat. A fusion beat occurs when an intrinsic event more or less coincides with a generated stimulation pulse. In this case there is no intrinsic activity and the LOG was not a result of a fusion beat but was due to a changed stimulation threshold of the heart tissue, and a stimulation threshold search is initiated.

In FIG. 1 during the threshold search the pre-programmed AV-interval is shortened to “AV-short” to override any intrinsic heart activity. The ventricular stimulation amplitude is successively stepped up by a predetermined amplitude step of e.g. 0.1-0.3 V and each unsuccessful ventricular stimulation pulse is followed by a back-up pulse. This is performed until the stimulation threshold is detected, i.e. capture is detected from the ventricular stimulation pulse, and the stimulation pulse amplitude is then set to a value that equals the stimulation threshold plus a working margin, e.g. 0.3 V. As an alternative the ventricular stimulation amplitude may start at an amplitude above the stimulation threshold and then successively be stepped down until non-capture occurs.

It should be noted that the threshold search according to the established technique disclosed in the above-mentioned U.S. Pat. No. 5,476,487 is performed by using a pre-programmed AVI shortened to “AVI-short” as indicated above.

U.S. Pat. No. 5,766,229 is related to a capture verification method and apparatus for an implantable pacemaker utilizing heart rhythm stability measurements to minimized the likelihood of fusion. The heart rhythm stability is measured by determining a mean cardiac interval and a standard deviation of the measured intervals. If those values are acceptable (stable heart rhythm) the capture verification is performed.

In “User's manual for Microny SR+”, pp 15-20, (Pacesetter AR, ordering no. 6324565 E50QE, published in 1995) is an “Autocapture Pacing system” described where the threshold search is conducted every eight hour. A threshold search is also automatically conducted when two consecutive stimuli have resulted in no capture.

The algorithm is disabled when noise is detected and for as 15 long as the noise is sensed.

Every threshold search requires the stimulation rate to be below the basic rate plus 20 min⁻¹.

As indicated above a stimulation threshold search might be 20 performed either in response of loss of capture or at predetermined times.

### SUMMARY OF THE INVENTION

The present invention is primarily directed to those threshold searches performed at a timely basis and not in response of detected loss of capture.

An object of the present invention is to provide an improved implantable heart stimulator adapted to perform stimulation threshold searches whereby a reliable result is obtained with a high degree of safety.

The above object is achieved in accordance with the invention in an implantable heart stimulator wherein a threshold search timing unit generates a timing signal at the same time or times each day, in order to initiate respective threshold searches at regular intervals, and wherein a threshold search unit initiates a stimulation threshold search at the specified time only if a stable condition index fulfills at least one predetermined criterion, the stable condition index being related to a result from at least one stability test, wherein the stability test is at least one of the following: a value of a detected evoked response signal following an emitted stimulation pulse does not indicate an emerging fusion beat, or a value of a detected evoked response signal following an emitted stimulation pulse does not indicate a fusion beat.

In a further embodiment of the invention, the stability test can be either of the above stability tests, or a stability test indicating that an impedance signal representing conditions inside the heart, in combination with the timing of an emitted stimulation pulse, does not indicate a fusion beat.

In an embodiment of the present invention a threshold search is performed at the same time(s) every day dependent on the natural variations of the stimulation threshold.

In the preferred embodiment of the invention described above, a stable condition index determined related to the condition of the heart and based on the result from a heart stability test that belongs to a first group of tests related to detection of parameters indicating emerging fusion beats.

According to another embodiment of the invention a stable condition index is determined related to the condition of the heart and based on result from a heart stability test that belongs to a second group of tests related to the spontaneous activity of the heart.

### DESCRIPTION OF THE DRAWINGS

FIG. 1 shows an IEGM illustrating the principles of a threshold search algorithm according to established standard prior art.

FIG. 2 is a schematic block diagram of an implantable heart stimulator according to the invention.

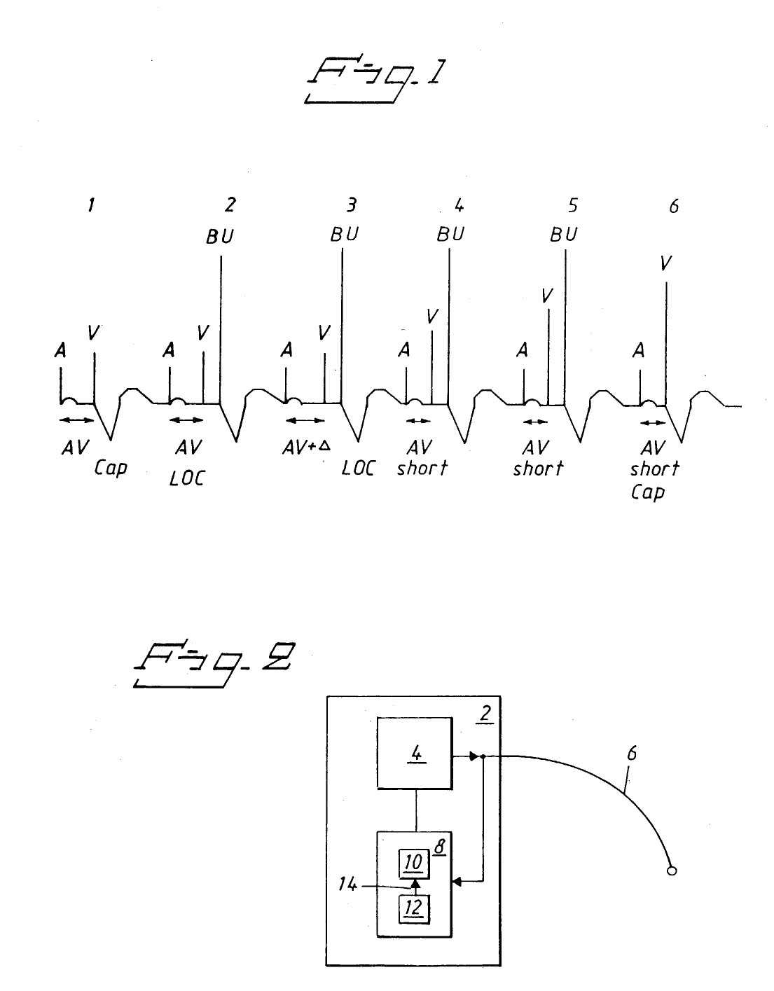

### DESCRIPTION OF THE PREFERRED EMBODIMENTS

FIG. 2 is a schematic block diagram of an implantable heart stimulator 2 according to the invention. The heart stimulator has a pulse generator 4 for generating stimulation pulses adapted to be applied to a heart via at least one electrode lead 6 and a control unit 8. The control unit 8 has a threshold search unit 10 for initiating a stimulation threshold search in order to determine a stimulation threshold of heart tissue and a threshold search timing unit 12.

The threshold search timing unit 12 generates a timing signal 14 at the same time every day to the threshold search unit 10 in order to initiate threshold searches. According to a preferred embodiment of the invention the timing signal is generated every 8 hours, e.g. 00, 08, 16, 24 hours. The timing signal need not be generated at a regular interval but instead at predetermined times, e.g. at 05 and 23 o'clock.

The generation of the timing signal is not affected by threshold searches performed due to loss of capture.

When a timing signal is generated and applied to the threshold search unit 10 a stable condition index is determined that is related to the condition of the heart and is based on the result from one or many heart stability tests.

According to a preferred embodiment of the invention the 25 heart stability test belongs to a first group of tests related to detection of parameters indicating emerging fusion beats.

A first heart stability test that belongs to the first group 30 of tests fulfils its criterion for an acceptable result if a value of a detected evoked response signal following an applied stimulation pulse does not indicate a fusion beat. This value may represent e.g. the integral of the evoked response signal measured during an interval, e.g. 15-55 ins, following the stimulation pulse as for instance as disclosed in PCT Application W099/65569.

A second heart stability test that also belongs to the first group of tests fulfils its criterion for an acceptable result if an impedance signal representing the conditions inside the heart in combination with the timing of the stimulation pulse does not indicate a fusion beat.

According to another preferred embodiment of the invention the stability test belongs to a second group of tests related to the spontaneous activity of the heart. Almost all implantable heart stimulators of today are equipped with a sensing means adapted to sense evoked and spontaneous electrical activity of the heart. To perform the tests of the second group such sensing means arranged in the control means is utilized.

A third heart stability test that belongs to the second group fulfils its criterion for an acceptable result if the number of inhibited stimulation pulses is lower than a predetermined number during a time interval immediately before a threshold search is to be performed. If, for example, the predetermined number is set to 1 and the time interval is set to 1 minute the result of the test is not acceptable if one stimulation pulse is inhibited less than one minute before the threshold search is to be performed.

A fourth heart stability test that belongs to the second group of tests fulfils its criterion for an acceptable result if the stimulation rate during the threshold search is at least 10 bpm higher than the spontaneous rate immediately before the threshold search (V-V interval shorter than R-R interval). In a heart stimulator working according to the VVI-mode the spontaneous rate may be tested by temporarily slow down stimulation rate before the search shall be performed (increase V-V interval). If the spontaneous rate is only 0-10 bpm less than the intended stimulation rate during threshold search the intended stimulation rate is increased by 5-20 bpm (decrease V-V interval). The reason for stimulating with a higher rate is to override any spontaneous activity. In a heart stimulator working according to a DDD mode of operation the AV/PV delay is temporarily prolonged to seek spontaneous conduction. The conducted PR/AR interval must be at least 10-70 ms longer than the intended AV/PV intervals to be used during the threshold search.

A fifth heart stability test that also belongs to the second group of tests fulfils its criterion for an acceptable result if no PVC is detected.

A sixth heart stability test that also belongs to the second group of tests fulfils its criterion for an acceptable result if no spontaneous intervals is shorter than the intended threshold search interval during the e.g. 50 cardiac cycles preceding a threshold search. In a heart stimulator operating in the DDD/VDD-mode a tracked spontaneous rate must not exceed the base rate with more than a programmable value. The absolute rate shall not exceed 120 bpm.

A seventh heart stability test that not belongs to any of the above-mentioned groups fulfils its criterion for an acceptable result if a sensor indicated stimulation frequency (rate responsive frequency) not exceeds a basic stimulation frequency with more than a predetermined value, preferably 10 or 20 beats per minute.

The stable condition index is determined by the control unit 8 in dependence of the result(s) from at least one heart stability test. This may be performed in many different ways. A straightforward way of determining the index is to generate a logic signal as a result of the heart stability test, whereas logic 1 indicates that the result of the stability test is acceptable and logic 0 indicates that the result not is acceptable. The logic signals are then applied at an AND-gate that generates a logic 1 as an output signal if all logical input signals are logical 1.

An alternative way of determining the stable condition index is to generate an analog value a result of each test, the analog value, ranging e.g. between 0-10, with values in the interval 7-10 acceptable. All values from the different tests are then added together and an interval of the sum defining an acceptable overall result may be determined.

The invention is equally applicable for single chamber heart stimulators as for double chamber heart stimulators. The tests used is dependent of the presently used stimulation mode (e.g. VVI, VDD, DDI, DDD).

If the threshold search not is performed due to a non-acceptable value from any of the tests all the tests are repeated within a predetermined period of time, e.g. within 5 minutes. The time for generation of timing signals by the threshold search timing unit is not altered in such cases.

Although modifications and changes may be suggested by those skilled in the art, it is the invention of the inventors to embody within the patent warranted heron all changes and modifications as reasonably and properly come within the scope of their contribution to the art.

1. Implantable heart stimulator (2) comprising a pulse generating means (4) for generating stimulation pulses adapted to be applied to a heart via at least one electrode lead (6), a threshold search timing means (12) that generates a timing signal at the same time(s) every day in order to initiate threshold searches at regular intervals, a control means (8) with threshold search means (10) for initiating a stimulation threshold search provided that a stable condition index fulfils predetermined criteria, wherein the stable condition index is related to the condition of the heart and is based on the result from at least one stability test, characterized in that said stability test is at least one of the following:
A value of a detected evoked response signal following an applied stimulation pulse does not indicate an emerging fusion beat.
A value of a detected evoked response signal following an applied stimulation pulse does not indicate a fusion beat.
An impedance signal representing the conditions inside the heart in combination with the timing of the stimulation pulse does not indicate a fusion beat.
The number of inhibited stimulation pulses is lower than a predetermined number during a time interval immediately before a threshold search is to be performed.

2. Heart stimulator according to claim 1 characterized in that said stability test is fulfilled if the integrated evoked response signal does not indicate emerging fusion.

3. Heart stimulator according to claim 1 characterized in that said stability test is fulfilled if the integrated evoked response signal does not indicate fusion.

4. Heart stimulator according to claim 1, characterized in that said timing signal is generated at a regular interval, e.g. every 8th hours.

5. Heart stimulator according to claim 1 or 2, characterized in that said heart stability test belongs to a second group of tests related to the spontaneous activity of the heart.

6. Heart stimulator according to claim 3, characterized in that a heart stability test fulfils its criterion for an acceptable result if the stimulation rate during the threshold search is at least 10 bpm higher than the spontaneous rate immediately before the threshold search (V-V interval shorter than R-R interval).

7. Heart stimulator according to claim 3, characterized in that the heart stability test fulfils its criterion for an acceptable result if no PVC is detected.

8. Heart stimulator according to claim 1, characterized in that a heart stability test fulfils its criterion for an acceptable result if a sensor indicated stimulation frequency not exceeds a basic stimulation frequency with more than a predetermined value, preferably 10-30 beats per minute.
